(19) United States
(12) Patent Application Publication
(10) Pub. No.: US 2014/0364833 A1
(43) Pub. Date: Dec. 11, 2014

(54) FETAL BLOOD RECOVERY SYSTEMS AND METHODS

(71) Applicants: Robert D. Christensen, Layton, UT (US); Vickie L. Baer, Clearfield, UT (US); Troy Jesse Orr, Draper, UT (US)

(72) Inventors: Robert D. Christensen, Layton, UT (US); Vickie L. Baer, Clearfield, UT (US); Troy Jesse Orr, Draper, UT (US)

(73) Assignee: INTERMOUNTAIN INVENTION MANAGEMENT, LLC, Salt Lake City, UT (US)

(21) Appl. No.: 14/118,667

(22) PCT Filed: May 17, 2012
(86) PCT No.: PCT/US12/38394

§ 371 Date: Aug. 27, 2014

Related U.S. Application Data
(60) Provisional application No. 61/488,010, filed on May 19, 2011.

(51) Int. Cl. A61M 1/02; A61M 39/22; A61M 1/00

(52) U.S. Cl. CPC A61M 1/02; A61M 1/0009; A61M 39/223; A61M 2202/0462; A61M 2240/00
USPC 604/507

(57) ABSTRACT

Methods and devices can be used to harvest fetal blood from a placenta. In some instances, the blood is used in laboratory testing so that no such blood is drawn from an infant for such testing. In other or further instances, harvested blood can be used in an autologous transfusion. Such procedures can eliminate the drawing of any blood directly from the neonate.

### TECHNICAL FIELD

The present disclosure relates generally to the recovery of fetal blood from a placenta.

### BRIEF DESCRIPTION OF THE DRAWINGS

The written disclosure herein describes illustrative embodiments that are non-limiting and non-exhaustive. Reference is made to certain of such illustrative embodiments that are depicted in the figures, in which:

FIG. 1A is a perspective view of an embodiment of an assembly configured for use in drawing fetal blood for testing and/or drawing fetal blood for autologous transfusion to an infant;

FIG. 1B is an exploded perspective view of the assembly of FIG. 1A;

FIG. 1C is a schematic representation of the assembly of FIG. 1A;

FIG. 2 is a perspective view of a stage of use of the assembly of FIG. 1A in which a blood vessel of a placenta is accessed and blood is drawn into a sampling blood receptacle for subsequent testing;

FIG. 3 is a cross-sectional view of the stage of use of the assembly that is shown in FIG. 2;

FIG. 4 is a perspective view of another stage of use of the assembly of FIG. 1A in which the sampling blood receptacle has been removed;

FIG. 5 is a perspective view of another stage of use of the assembly of FIG. 1A in which blood is drawn from the blood vessel into a transfusion blood receptacle for subsequent use in a transfusion;

FIG. 6 is a perspective view of another stage of use of the assembly of FIG. 1A in which the assembly has been withdrawn from the blood vessel and a needle has been removed from the assembly;

FIG. 7 is a perspective view of another stage of use of the assembly of FIG. 1A in which autologous blood is transfused from the transfusion blood receptacle into an infant;

FIG. 8 is a perspective view of an embodiment of a kit that is compatible with certain methods disclosed herein;

FIG. 9A is a perspective view of another embodiment of an assembly configured for use in drawing fetal blood for testing and/or drawing fetal blood for autologous transfusion to an infant;

FIG. 9B is an exploded perspective view of the assembly of FIG. 9A;

FIG. 10 is an exploded perspective view of an embodiment of a stopcock that is compatible with the assembly of FIG. 9A;

FIG. 11A is a perspective view of the stopcock of FIG. 10 in a first operational state;

FIG. 11B is a perspective view of the stopcock of FIG. 10 in a second operational state;

FIG. 12 is a perspective view of a stage of use of the assembly of FIG. 9A in which a blood vessel of a placenta is accessed and blood is drawn into a sampling blood receptacle for subsequent testing;

FIG. 13 is a perspective view of another stage of use of the assembly of FIG. 9A in which the sampling blood receptacle has been removed and in which blood is drawn from the blood vessel into a transfusion blood receptacle for subsequent use in a transfusion; and

FIG. 14 is a perspective view of another stage of use of the assembly of FIG. 9A in which autologous blood is transfused from the transfusion blood receptacle into an infant.

### DETAILED DESCRIPTION

One devastating occurrence in modern neonatal intensive care is intraventricular hemorrhage (IVH). Hemorrhages of this variety virtually always occur during the three days following birth of very low birth weight (VLBW) infants, which are generally defined as infants whose mass is less than 1500 grams. When such hemorrhages are graded as severe (e.g., grade 3 or 4) they generally presage significant, life-long neurodevelopmental handicaps. It is believed that approximately 25 percent of very low birth weight neonates develop an IVH, and about 10 percent of these develop a severe IVH. Although IVH is a multi-factorial condition, in which some of the associated factors are not amenable to correction after birth (e.g., genetic predisposition, the fragile make up of the vascular structure of the germinal matrix, intrauterine complications), other factors may be addressed by the methods and devices disclosed herein, as further discussed below.

For example, phlebotomy followed by red blood cell transfusions using donor blood can cause IVH. Without being limited by theory, it is noted that term infants have pericytes surrounding the fragile capillaries in the germinal matrix of the brain. These are supporting cells giving the capillaries strength and preventing rupture. However, pericytes have not yet formed surrounding capillaries in the premature germinal matrix. Red blood cells can transverse the fragile capillaries in line (i.e., in single file) and can require both deformability and the release of nitric oxide synthase to relax the capillaries for the red blood cells to pass. Banked donor red blood cells can develop a storage lesion involving poor deformability and loss of nitric oxide synthase. The storage lesion can result in a reduced ability of the transfused red blood cells to adequately navigate and dilate the capillaries. If red blood cells get stuck in the unsupported capillaries, pressure can build up and the wall of the capillary can rupture, instituting a brain hemorrhage. It is noted that when a premature neonate is admitted to the NICU, the initial blood tests drawn for laboratory analysis can equate to about 10 percent or more of the blood volume of the neonate, which can contribute to the need for red blood cell transfusions.

Accordingly, it can be desirable to reduce or eliminate the need for red blood cell transfusions in neonates and/or reduce or eliminate the use of donor blood where such transfusions take place. Certain embodiments disclosed herein can achieve one or more of these desirable goals. For example, some embodiments can reduce or eliminate the need for drawing blood from the neonate for initial blood tests, and other or further embodiments can substitute autologous blood transfusions, in which the infant's own fetal blood is used, for standard donor blood transfusions.

As further discussed below, in some embodiments, a method for drawing blood for post-delivery tests comprises drawing all or a substantial portion of blood needed for laboratory tests (e.g., admission blood tests) from fetal blood in the placenta. For example, fetal blood can be drawn from the placenta immediately following placental delivery. Such procedures can eliminate the drawing of any blood directly from the neonate. In other or further embodiments, fetal blood is harvested from the placenta for purposes of autologous transfusion. In some embodiments, the harvested fetal blood is used in such a transfusion immediately or shortly after its collection. Accordingly, certain embodiments disclosed herein can significantly reduce donor blood transfusions into VLBW infants during their first days after birth, and thereby reduce the prevalence of brain hemorrhages.

Other uses and advantages of the methods, systems, and devices disclosed herein are also contemplated. Accordingly, although certain embodiments may provide particular advantages in the context of premature infants, wider applications are possible. For example, fetal blood harvesting such as disclosed herein may be performed for term infants. The use of fetal blood drawn from the placenta for initial blood tests can eliminate the discomfort associated with drawing the blood directly from the infant. Similarly, obtaining fetal blood for an autologous transfusion may be useful in a variety of situations for term infants (e.g., surgery shortly after birth, or those with known antenatal anemia, abruption, or other hemorrhage).

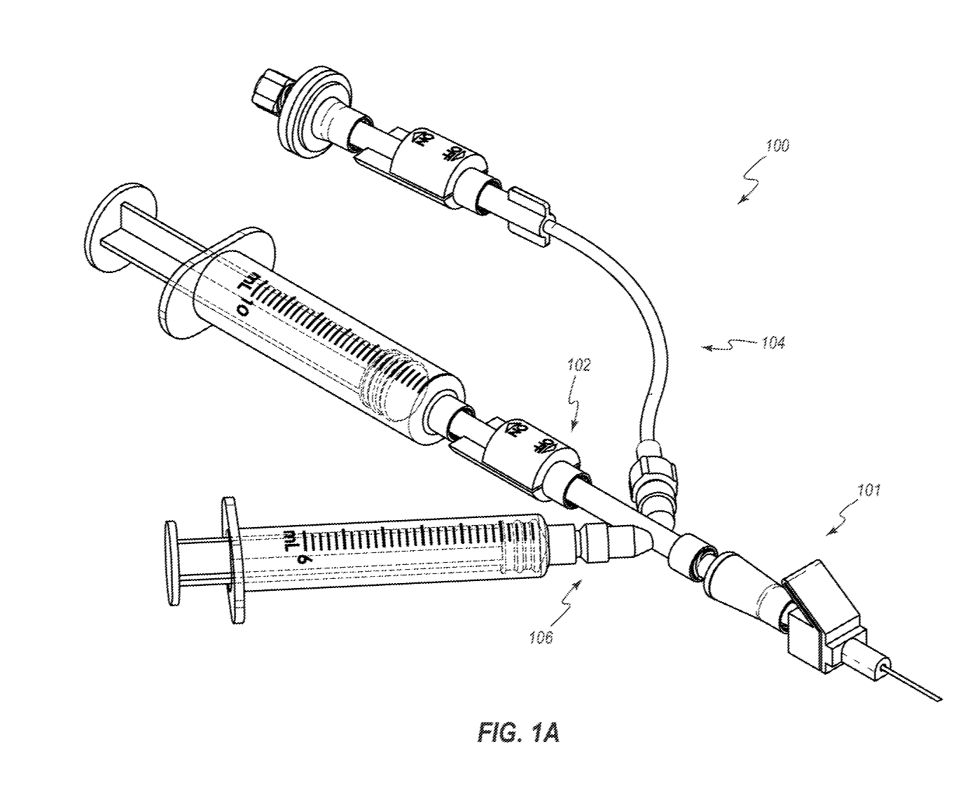

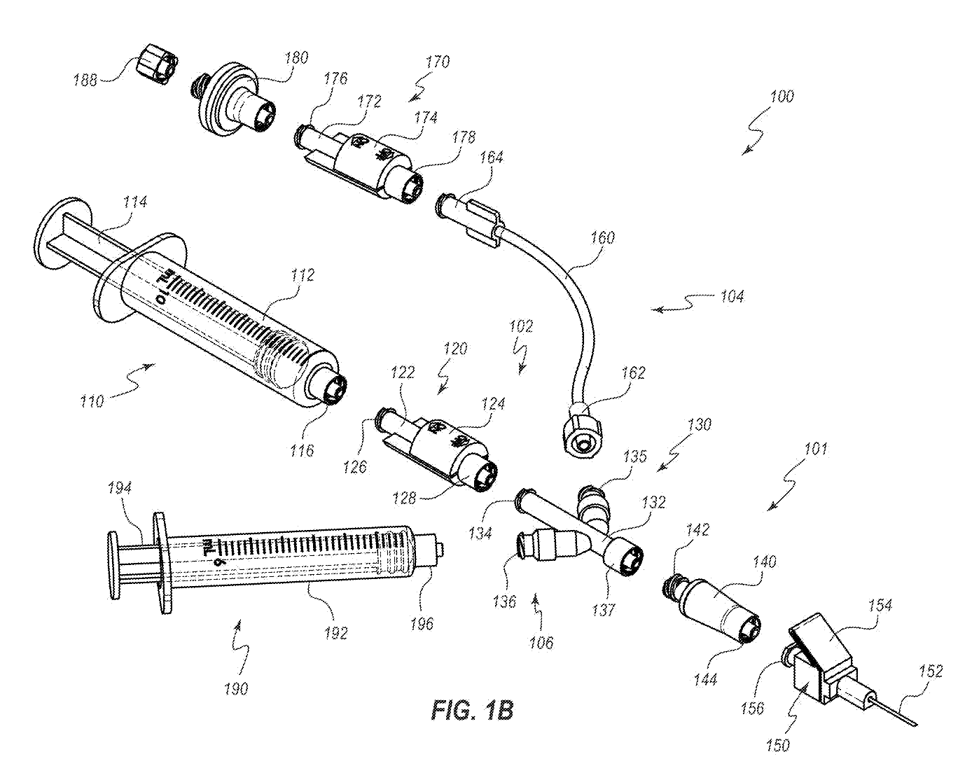

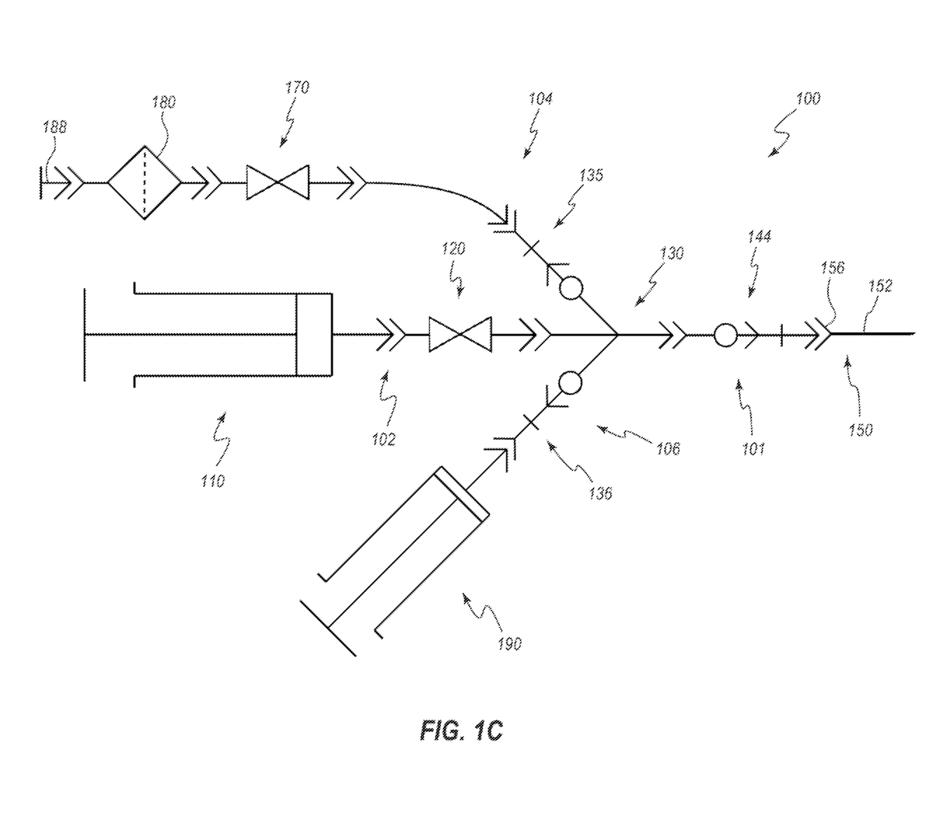

FIGS. 1A-1C illustrate various views and depictions of an embodiment of a blood harvesting assembly 100 that can be used in collecting fetal blood from a placenta. In FIG. 1A, the assembly 100 is in an assembled, pre-use state; in FIG. 1B, the assembly 100 is in a disassembled (or pre-assembled) state; in FIG. 1C, the assembly 100 is depicted schematically. As shown in FIG. 1A, the assembly 100 can comprise multiple fluid channels, fluid lines, or fluid paths 101, 102, 104, 106. A collection fluid path 101 defines a passageway through which fetal blood flows after it has been extracted from a placenta, as further discussed below. In the illustrated embodiment, additional fluid paths include a transfusion fluid path 102, a delivery fluid path 104, and a sampling fluid path 106, each of which defines a passageway suitable for permitting fetal blood to flow therethrough for a respective purpose, as discussed below.

With reference to FIGS. 1B and 1C, the collection fluid path 101 can include a puncturing, insertion, or needle device 150 that is configured to be introduced into a blood vessel so as to withdraw blood therefrom. The illustrated needle device 150 includes a needle 152, a housing 154, and a connector 156. The needle 152 can be of any suitable variety, including those commonly used in phlebotomy applications. The housing 154 can be of any suitable variety, and in some embodiments, can be configured to draw the needle 152 therein after use of the needle 152 so as to prevent accidental sticks. Many suitable needle retraction technologies that can be used with the housing 150 in this manner are known. The connector 156 can also be of any suitable variety. In the illustrated embodiment, the connector 156 defines a standard female luer lock arrangement.

The collection fluid path 101 can further include a valved connector 140. In the illustrated embodiment, the valved connector 140 comprises a closed male connector. In particular, the connector 140 comprises a male connector 144 at one end and a female connector 142 at an opposite end. The male connector 144 is a “closed” or “closable” connector, meaning that the connector 144 comprises a valving feature that is configured to be in an open state when the connector 156 is coupled with the male connector 144 and is configured to automatically transition to a closed state when the connector 156 is decoupled from the male connector 144. Many suitable valving male connectors 144 are known. In the illustrated embodiment, the male connector 144 includes a standard male luer lock arrangement for coupling with the female luer lock arrangement of the connector 156.

In FIG. 1C, the valving male connector 144 is depicted with a symbol that indicates it is both an “auto-closing” or “auto-shutoff” connector and a male connector. In contrast, the connector 156 of the needle device 150 is shown only as a female connector. Since the connector 156 is “open” (e.g., does not include an automatically closable valving feature), it merely includes a leftward-opening chevron, indicating that it is a female connector that can receive the rightward-pointing chevron of the male connector 144. Due to the automatic closing or valving capabilities of the connector 144, the collection fluid path 101 can close automatically when the needle device 150 is removed from the assembly 100. Such automatic valving can cause the assembly 100 to be a closed system, or can maintain the assembly 100 as a closed system. As used herein, the term “closed system” is a broad term that refers generally to a system that defines a closed environment that is segregated, separated, or otherwise sealed from a surrounding environment. In some instances, phlebotomy apparatuses and phlebotomy systems can desirably be closed systems, which can reduce or eliminate contamination of blood after the blood has been drawn into the system. When in the open state, the connector 144 can permit fluid communication between the needle 152 and portions of the collection fluid path 101 that are at an opposite side of the connector 144, whereas when the needle device 150 is removed from the assembly 100 and the connector 144 is in the closed state, the connector 144 seals the collection fluid path 101 from an environment that surrounds the assembly 100.

With reference again to FIGS. 1B and 1C, the valved connector 140 can be connected to a branched connector (e.g., tri-port connector), or manifold connector 130. In the illustrated embodiment, the manifold connector 130 comprises an open male connector 137, an open female connector 134, and two auto-shutoff or closed female connectors 135, 136. The fluid path 101 branches into the collection fluid path 106, the transfusion fluid path 102, and the delivery fluid path 104 at the manifold connector 130. A housing portion 132 of the connector 130 can be open such that all of the fluid paths 101, 102, 104, 106 are in fluid communication with each other within the housing 132.

At least a portion of the collection fluid path 106 is defined by the closed female connector 136. A sampling syringe 190 can be coupled to the female connector 136 via a male connector 196. The syringe 190 can include a blood receptacle 192 and a plunger 194 that is movable relative to the blood receptacle 192. In other embodiments, different blood receptacles may be used in the place of the syringe 190. As further discussed below, in some embodiments, the sampling syringe 190 can be used to collect fetal blood for use in laboratory testing. The blood collection can take place when the assembly 100 is in a sample blood harvesting mode, which is discussed further below. In some embodiments, it can be desirable for the syringe 190 to be selectively detachable from the assembly 100 so that the harvested blood within the syringe 190 can readily be transported to a desired location (e.g., a blood laboratory).

In certain embodiments, when the syringe 190 is coupled with the connector 136, the connector 136 is in the open state and permits fluid communication between the syringe 190 and the sampling fluid path 106. When the syringe 190 is decoupled from the assembly 100, the connector 136 can automatically transition to the closed state, and the connector 136 can thereby seal the sampling fluid path 106 from an environment that surrounds the assembly 100.

In addition to a branch of the manifold connector 130, the transfusion fluid path 104 can include a valved connector 120. In the illustrated embodiment, the valved connector 120 is a shut-off valve connector 122, that includes a female connector 126 and a male connector 128 at opposite ends thereof. A transfusion syringe 110, which includes a blood receptacle 112 and a plunger 114, can be connected to the connector 126 via a connector portion 116.

The shut-off valve connector 122 includes a manually operable switch or actuator 124 that can be moved so as to transition the connector 122 between an open orientation and a closed orientation. When in the closed orientation, the connector 122 prevents fluid communication between opposite ends of the connector 122—e.g., can prevent fluid communication between the manifold connector 130 and the transfusion syringe 110. When in the open orientation, the connector 122 permits fluid to pass therethrough. Accordingly, when the assembly 100 is in an appropriate mode, pulling back on the plunger 114 can draw fetal blood through the transfusion fluid path 102 and into the blood receptacle 112 of the syringe 110. Such a mode may be referred to as a transfusion blood harvesting mode, which is discussed further below.

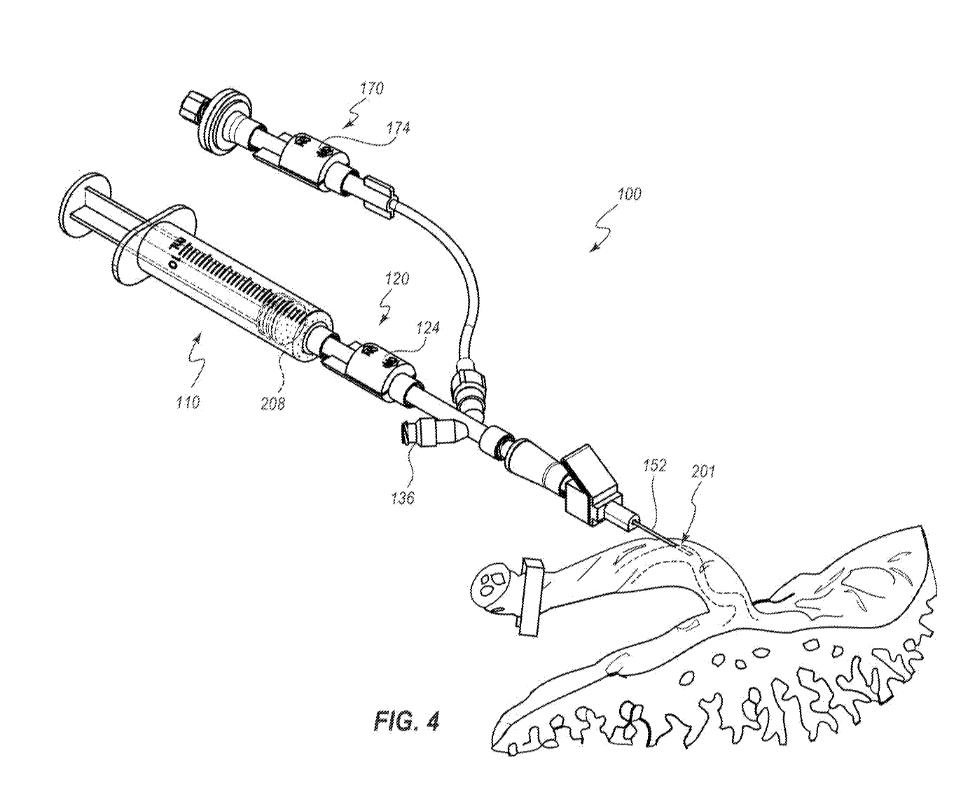

In some embodiments, the transfusion syringe 110 can be pre-loaded with an anticoagulant 208 (see FIG. 4). In certain of such embodiments, it can be advantageous to ensure that the shut-off valve connector 122 is in the closed state when blood is being drawn into the sampling syringe 190, as this can prevent the anticoagulant 208 from being drawn from the transfusion syringe 110 into the sampling syringe 190. The presence of anticoagulant 208 in the fetal blood could be detrimental to certain tests that may be run on blood that is subsequently removed from the syringe 190.

The delivery fluid path 104 can include the connector 135, as previously mentioned, as well as a tube 160 that includes connectors 162, 164 at either end, a valved connector 170, and/or a filter 180. The valved connector 170 can resemble the valve connector 120 described above. For example, in the illustrated embodiment, the valved connector 170 is a shut-off valve connector 172, which includes a female connector 176 and a male connector 178 at opposite ends thereof and a manually operable actuator 174. When in a closed orientation, the connector 172 prevents fluid communication between opposite ends of the connector 172—e.g., can prevent fluid communication between the manifold connector 130 and the filter 180. When in the open orientation, the connector 172 permits fluid to pass therethrough. Accordingly, depressing the plunger 114 can force fetal blood through the transfusion fluid path 102 and through the delivery path 104 when the assembly 100 is in an appropriate operational mode. Such a mode is referred to herein as a transfusion mode, which is discussed further below.

The filter 180 can be of any suitable variety, and may be configured to prevent particles larger than a predetermined size from being transfused into an infant. For example, various embodiments, the filter 180 can be configured to prevent passage of particles larger than about 40, 150, 170, 200, or 250 microns, while readily permitting the passage of blood cells. In some embodiments, a cap 188 may be connected with filter 180 when the assembly is in a pre-use state.

For any of the connector arrangements discussed above, the specific male-female relationships recited are not intended to be limiting. For example, in some embodiments, the connector 156 is instead a male connector, whereas the connector 144 is a complementary closed or valved female connector. Additionally, connection interfaces other than luer-type interfaces are possible. Moreover, in some embodiments, such as certain embodiments in which a particular component is not intended for removal from the assembly, each connector in a complementary connector pair may be “open.” For example, in some embodiments, both the connector 135 and the connector 162 are open connectors that remain connected to each other at all times during use of the assembly 100, thereby maintaining the entirety of the delivery fluid path 104 coupled to the assembly 100 throughout all stages of a harvesting and transfusion procedure.

Moreover, in some embodiments, many components of the assembly 100 may be permanently affixed and/or integrally connected to each other. For example, in some embodiments, the connector 135 and the connector 162 of the fluid path 104 may be replaced by a single, integrally formed component. As another example, the needle device 150 and the valved connector 140, and/or the valved connector 140 and the manifold connector 130 portions of the fluid path 101 can be permanently affixed to each other, and housing portions of these components may be formed of a single piece of integrally formed material. Certain of such embodiments may reduce the deadspace of the collection fluid path 101 and or may exhibit other distinguishing or desirable features relative to the more modular embodiment depicted in FIG. 1B. Similar arrangements are possible for the other fluid paths 104, 106. Any other suitable arrangements of the assembly 100 are also contemplated.

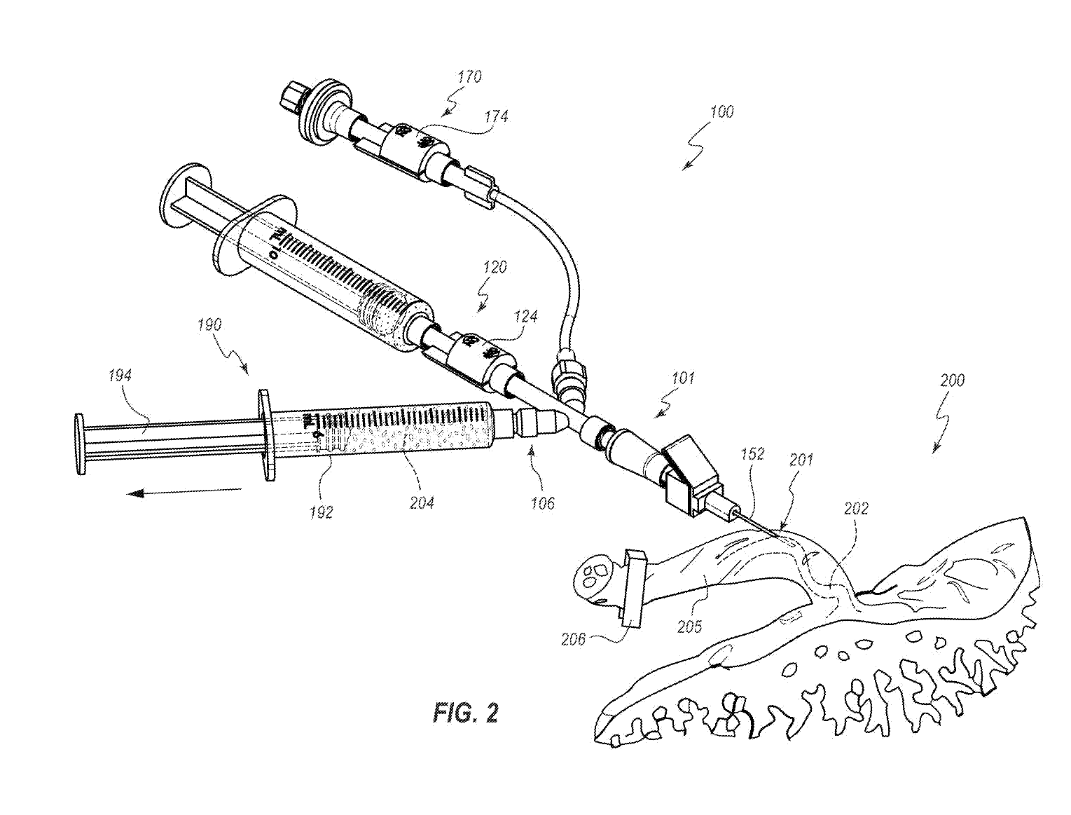

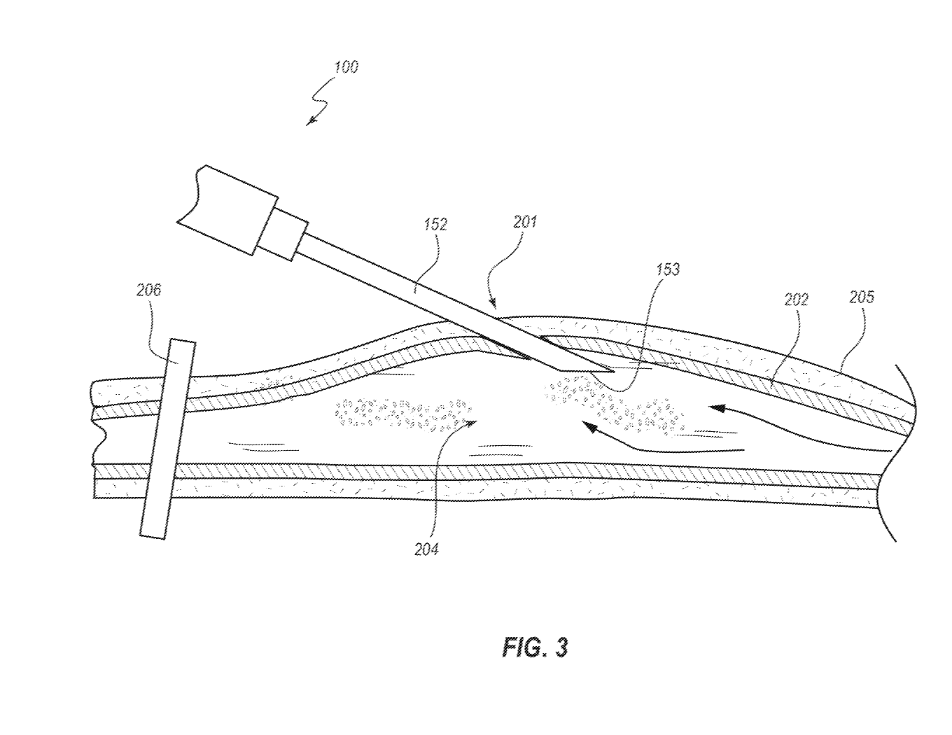

FIGS. 2 and 3 illustrate an early stage in a method of using the assembly 100. As shown in FIG. 2, a delivered placenta 200 includes a portion of an umbilical cord 205 attached thereto. The umbilical cord 205 has been clamped closed via any suitable clamp 206. Such clamping can isolate fetal blood that is within the placenta 200 from an environment that is exterior to the placenta 200, which can render the fetal blood in the placenta 200 and the recovery system a “closed system.” Although not shown in the drawings, in some methods, the umbilical cord 205 is clamped via the clamp 206 before the umbilical cord 205 is cut. For example, in some methods, the clamp 206 is attached to the umbilical cord 205 in the position shown in FIGS. 2 and 3 and an additional clamp (not shown) that is positioned on the umbilical cord 205 at a position closer to the infant. Thereafter, the umbilical cord 205 is cut at a position between the clamps. Such a procedure can maintain sterility of blood within the placenta 200.

In some embodiments, a portion of the placenta 200 and/or the umbilical cord 205 has been disinfected prior to insertion of the needle 152 into the placenta 200 at an insertion site 201. For example, in some embodiments, a surface of the placenta 200 and/or a surface of the umbilical cord 205 are swabbed with providone iodine and allowed to dry for about 60 seconds. For ease of discussion, certain references herein to the “placenta” may include one or more of the placenta 200 and the portion of the umbilical cord 205 that remains attached to the placenta 200. Accordingly, it may be said that a surface of the placenta 200 at which an insertion site or access site 201 is to be formed is disinfected prior to inserting the needle 152 into the placenta 200 at the insertion site 201, even where the access site 201 is in fact located at the umbilical cord 205 remnant.

With continued reference to FIGS. 2 and 3, the needle 152 can be inserted into any suitable blood vessel 202 so as to access the fetal blood 204. In some embodiments, the umbilical vein is accessed. As is well, known, the umbilical vein can extend through the umbilical cord 205 and along a surface of the placenta 200. In the illustrated method, the blood vessel 202 is accessed within the umbilical cord 205 region. A tip 153 of the needle 152 can be inserted in a bevel-down orientation, which can facilitate blood harvesting. For example, the wall of the umbilical vein can be very pliable, and thus a side of the wall that is opposite from the insertion site 201 may be readily pulled into the opening at the tip 153 of the needle 152 and thereby obstruct or clog the needle 152, if a portion of the tip 153 is close to the opposing sidewall when blood is being drawn into the needle 152. The bevel of the tip 153 may be substantially parallel to an opposing sidewall. In some approaches, the umbilical vein is “tented” in an arrangement such as that shown in FIG. 3 so as to further space the tip 153 of the needle 152 from the opposing sidewall of the umbilical vein. The “tented” arrangement can permit blood to pool in the enlarged area. The fetal blood 204 may be withdrawn slowly so as to reduce the forces on the opposing sidewall of the vein that would cause it to move into proximity to the tip 153. Due to clamping of the umbilical cord 205, the vasculature of the placenta 200 may not be vented to the surrounding environment, such that a vacuum-like effect may arise within the vasculature as fetal blood is withdrawn therefrom. This can contribute to the natural tendency of the sidewall of the blood vessel 202 to move toward and/or stop up the tip 153 of the needle 152 and/or otherwise cause the pliable blood vessel 202 to close.

With reference to FIG. 2, the assembly 100 is in the sample blood harvesting mode. In particular, the actuators 124, 174 of the valved connectors 120, 170 are each in a closed orientation. With the needle 152 having been inserted into the blood vessel 202, as just discussed, the plunger 194 is withdrawn relative to the blood receptacle 192, and fetal blood 204 is drawn through the collection fluid path 101 and the sampling fluid path 106 into the blood receptacle 192. In some methods, the sample fetal blood 204 is collected in the syringe 190 before blood is collected in the transfusion syringe 110. In some instances, this is because it may be more desirable to ensure that blood tests are performed on the fetal blood than to preserve fetal blood for transfusion, should an insufficient amount of harvestable blood be available for both uses.

With reference to FIG. 4, the syringe 190 can be decoupled from the assembly 100 and the blood collected therein can be tested using any suitable procedure or procedures. In the illustrated embodiment, the assembly 100 remains in the sample blood harvesting mode, as the orientations of the actuators 124, 174 remains unchanged. Additionally, the needle 152 remains inserted through the single insertion site 201.

As previously noted, the transfusion syringe 110 includes an anticoagulant 208 therein. In some embodiments, the transfusion syringe 110 may be pre-loaded with the anticoagulant 208 and attached to the assembly 100 when the assembly 100 is in a pre-use state. In other embodiments, the transfusion syringe 110 may be loaded with anticoagulant 208 at any point prior to transition of the assembly 100 into the transfusion blood harvesting mode. For example, in some embodiments, the transfusion syringe 110 is loaded with anticoagulant and attached to the assembly 100 at the stage illustrated in FIG. 4. Additionally, it is noted that in some embodiments the transfusion syringe 110 may be larger than the sample syringe 190. For example, the transfusion syringe 110 may be a 10 milliliter syringe, whereas the sample syringe 190 may be a 5 milliliter or a 6 milliliter syringe.

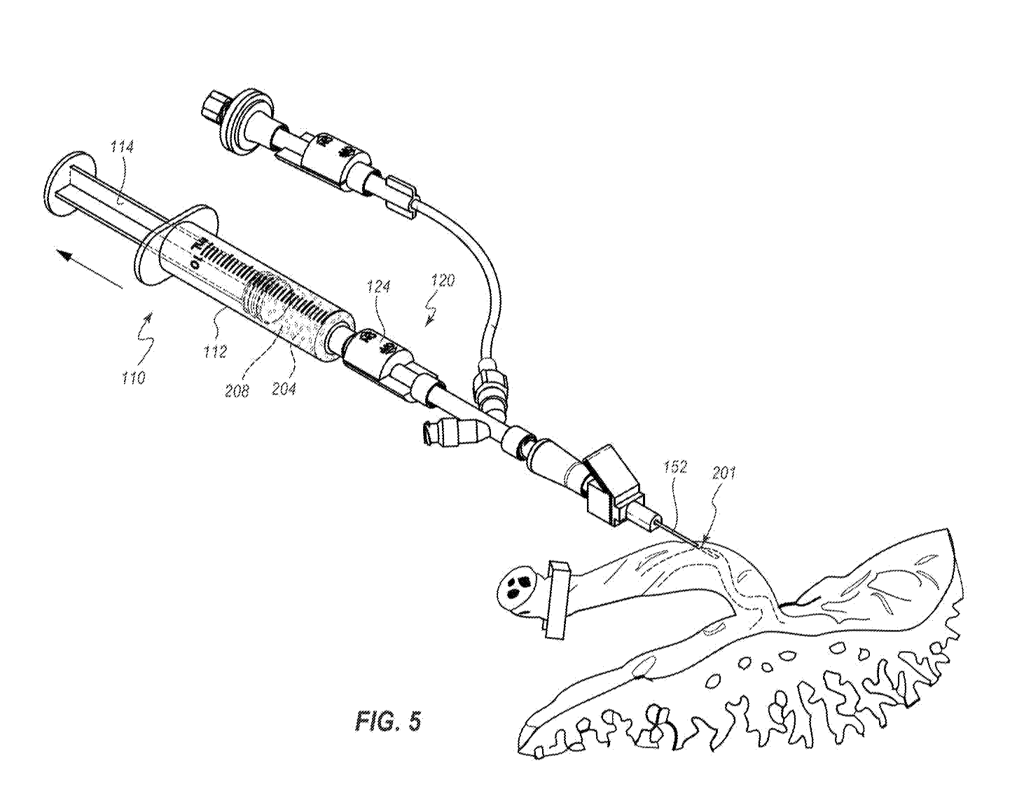

FIG. 5 illustrates a later stage of the illustrative method in which the assembly 100 has been transitioned into the transfusion blood harvesting mode. In particular, the actuator 124 has been moved so as to transition the valved connector 120 into the open state. The plunger 114 can be withdrawn relative to the blood receptacle 112, which can draw fetal blood 204 into the blood receptacle 112 where it mixes with the anticoagulant 208.

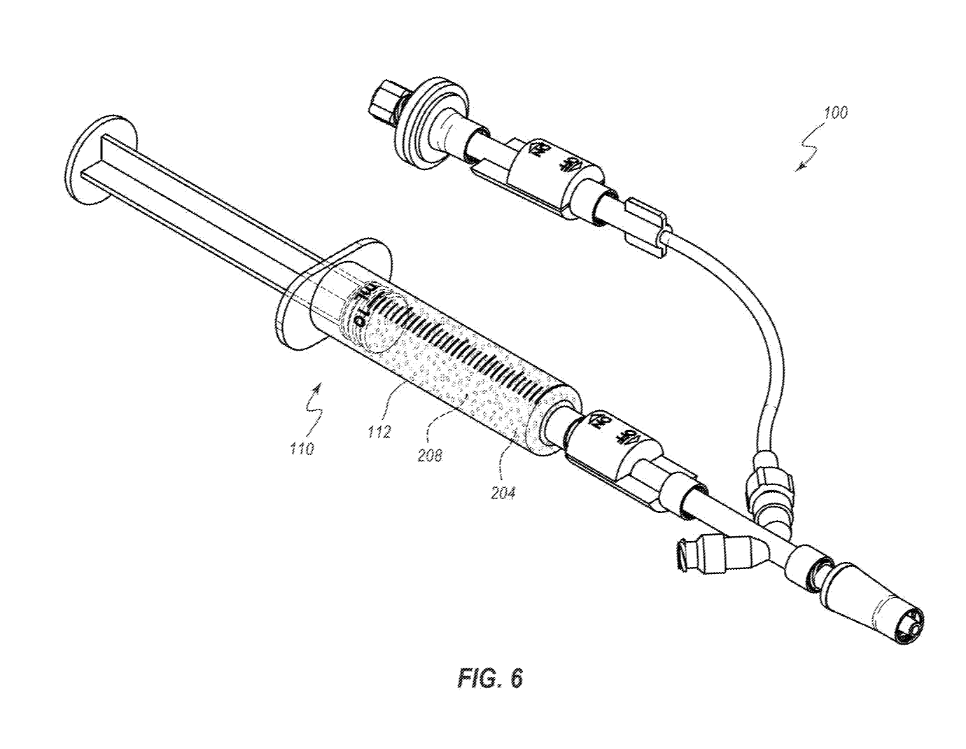

FIG. 6 illustrates another stage of the illustrative method in which the needle device 150 has been removed from the assembly 100. In this and/or other stages, the syringe 110 can be rotated or otherwise agitated so as to further mix the collected blood 204 with the anticoagulant 208.

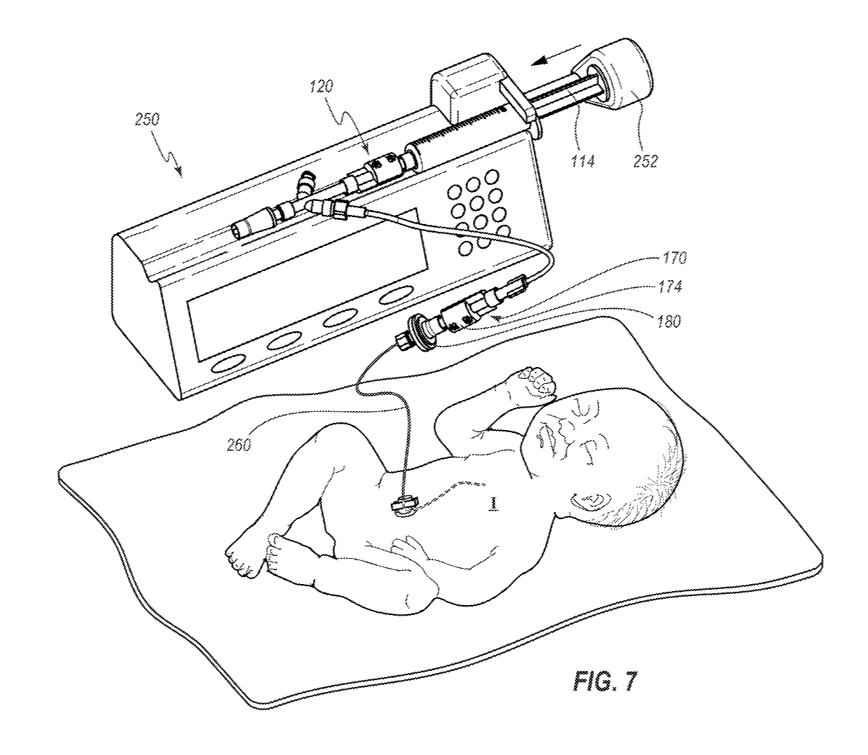

FIG. 7 illustrates another stage of the illustrative method in which the assembly 100 has been transitioned into the transfusion mode. In particular, the valved connector 120 remains in the open state, and the actuator 174 has been moved so as to transition the valved connector 170 into the open state. The assembly 100 has been coupled with a catheter 260 through which blood can be delivered to an infant I.

The assembly 100 is loaded onto an automatic syringe pump 250. The pump 250 may be programmable so as to transfuse blood to the infant I in any suitable manner. The pump 250 can include a dispensing arm 252 that is configured to depress the plunger 114 at a desired rate so as to deliver the blood 204 to the infant I at the desired rate.

In some methods, the transfusion procedure depicted in FIG. 7 may be initiated once the hemoglobin level of the infant I has dropped below a predetermined threshold level. For example, some protocols may call for a transfusion (e.g., a red blood cell transfusion) once an infant's hemoglobin level drops below this threshold level. In other methods, the transfusion procedure may be initiated when the infant's hemoglobin level is above the threshold level. Such a “preemptive” infusion may prevent the infant's hemoglobin level from ever dropping below the predetermined threshold.

In some instances, the transfusion procedure can be started immediately or very soon after the transfusion fetal blood has been collected from the placenta 200. For example, the fetal blood may not be placed in sort of storage prior to its use in a transfusion. In various instances, the transfusion procedure may begin no more than about 5, 10, 15, 20, or 30 minutes after the transfusion syringe 110 has been charged with fetal blood. In other or further instances, it can be desirable to complete the transfusion procedure within a certain timeframe of the harvesting of the fetal blood. For example, in various instances, the transfusion procedure may be completed no more than about 1, 2, 3, 4, 5, or 6 hours after the transfusion syringe 110 has been charged with fetal blood.

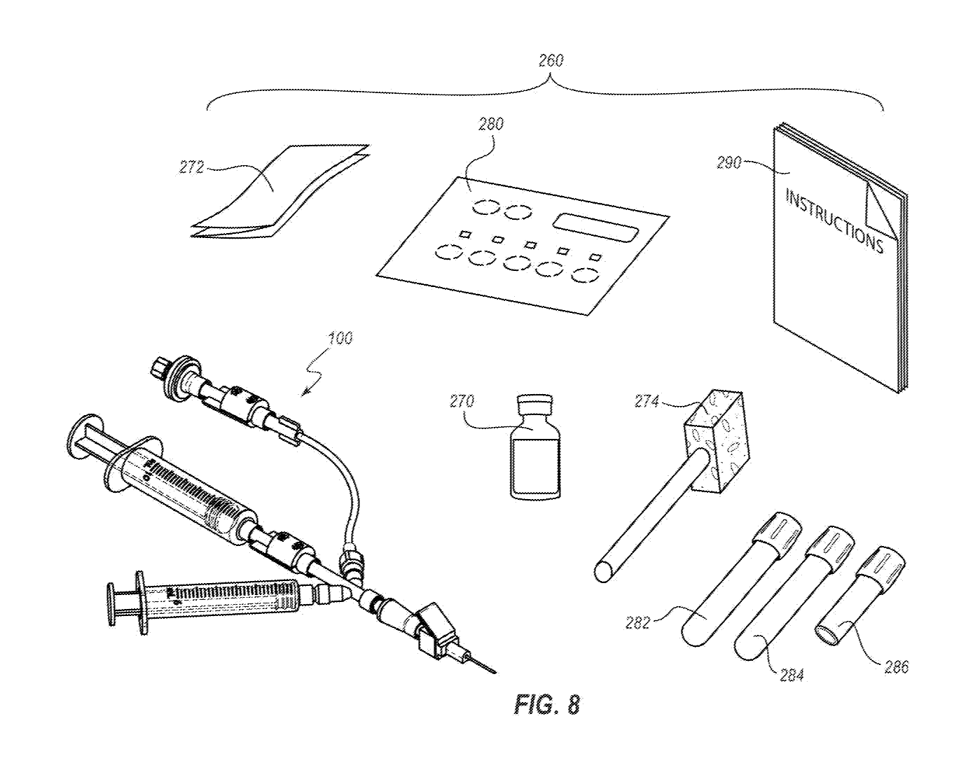

FIG. 8 illustrates an embodiment of a kit 260 that can include materials and/or devices that can be used in harvesting fetal blood and/or in testing at least a portion of the harvested blood. The kit 260 can include any embodiment of a blood harvesting assembly (e.g., any of the assemblies 100) discussed herein. Additional items may also be included in the kit 260. For example, in some embodiments, the kit 260 can include one or more of an anticoagulant 270, a sterile paper towel 272, and a disinfecting swab 274 (e.g., a packaged providone-iodine-saturated swab). As previously discussed, in some embodiments, an anticoagulant may be preloaded in a blood receptacle of the assembly 100.

In other or further embodiments, the kit 260 can include a metabolic screen 280, which may be used in a known manner with a portion of the collected fetal blood. In some instances, a different version of the metabolic screen 280 may be included with the kit 260, as such screens can typically vary from state to state. However, in other embodiments, the kit 260 does not include a metabolic screen 280.

In other or further embodiments, the kit 260 can include any suitable combination of tubes 282, 284, 286 for receiving fetal blood for culture, umbilical venous blood gas analysis, coagulation tests, complete blood cell counts, metabolic screening tests, and/or any other suitable laboratory tests. The kit 260 may be suitable for rapid distribution and use, as certain of such tubes may outdate rapidly (e.g., have a shelf life of a few months). The kit 260 may also include different combinations or types of tubes 282, 284, 286, depending on the hospital to which it is distributed, since the tubes used in certain protocols often vary from hospital to hospital. However, in other embodiments, the tubes are not included in the kit 260.

The kit 260 may also include instructions 290 for using the blood harvesting assembly and/or any other components of the kit 260. The instructions 290 can include directions for performing any and/or all of the steps of any suitable method for harvesting blood using the assembly, such as any of the procedures discussed above. In other or further embodiments, the instructions 290 may provide directions for accessing such directions for use. For example, the instructions may list a web address, a mailing address, and/or a telephone number that can be used to locate instructions for harvesting fetal blood. One or more of the foregoing items can be included in and/or on (e.g., in the case of the instructions) packaging (not shown) in which the kit 260 is distributed.

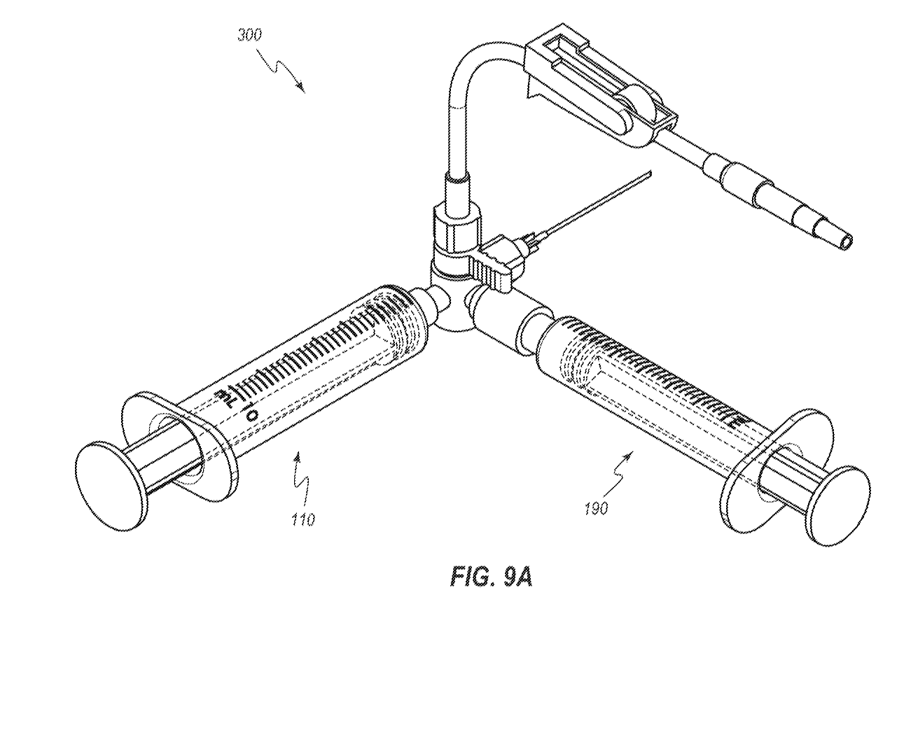

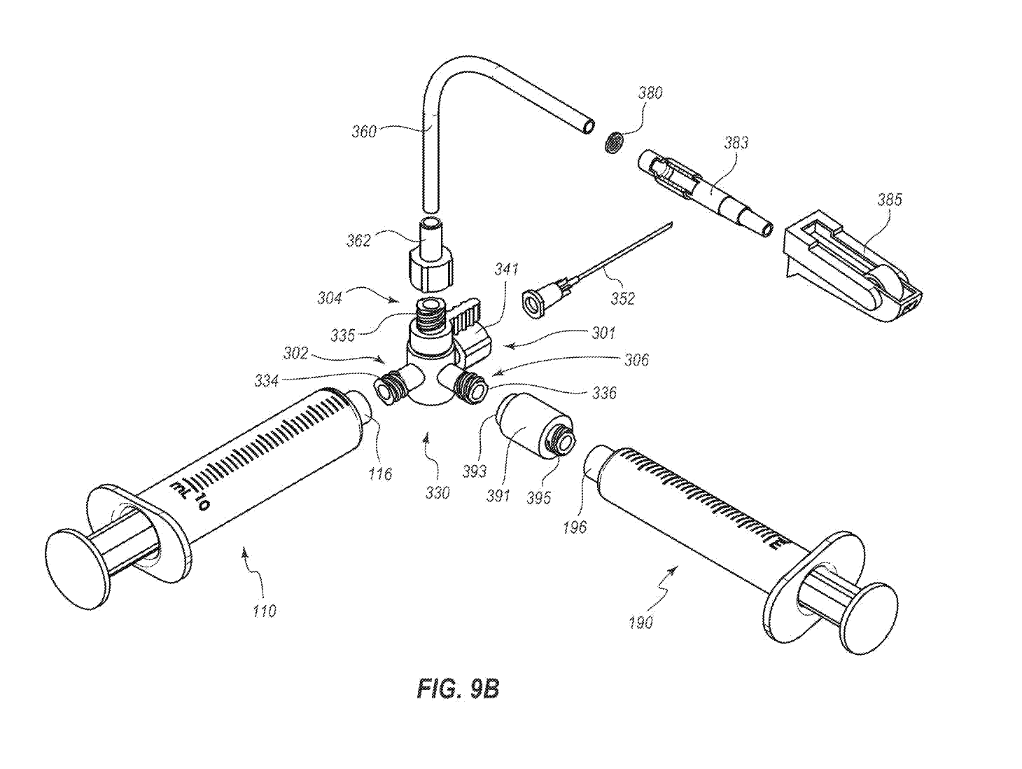

FIGS. 9A-14 illustrate another embodiment of a blood harvesting assembly 300, components thereof, and methods of using the same that can resemble the blood harvesting assembly 300, components thereof, and methods of using the same discussed above in certain respects. Accordingly, like features may be designated with like reference numerals, with the leading digits incremented to “3.” Relevant disclosure set forth above regarding similarly identified features thus may not be repeated hereafter. Moreover, specific features of the assembly 300 may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the assembly 300. Any suitable combination of the features and variations of the same described with respect to the assembly 100 can be employed with the assembly 300, and vice versa.

With reference to FIGS. 9A and 9B, the assembly 300 can include and/or can be configured for use with one or more syringes 110, 190 in manners such as described above. The assembly 300 can include a valve (e.g., a valve connector), a manifold connector, or a stopcock 330, which can define at least a portion of each of a collection fluid path 301, a sampling fluid path 306, a transfusion fluid path 302, and a delivery fluid path 304.

In the illustrated embodiment, the collection fluid path 301 includes a needle 352 that is connected to the stopcock 330 via a connector 341. The sampling fluid path 306 includes an open connector 336 that can be connected with an open connector portion 393 of a valve member 391. The valve member 391 can include a closed connector 395 that can be selectively coupled to the connector portion 196 of the syringe 190. The transfusion fluid path 302 can include an open connector 334 defined by the stopcock 330, which can be selectively connected to the connector portion 116 of the syringe 110. The delivery fluid path 304 can include an open connector 335 defined by the stopcock 330, which can be connected with tubing 360 via a connector 362. The delivery line 360 can further include a filter 380, a connector 383, and a clamp 385 (e.g., a roller clamp). The clamp 385 can selectively close the tubing 360.

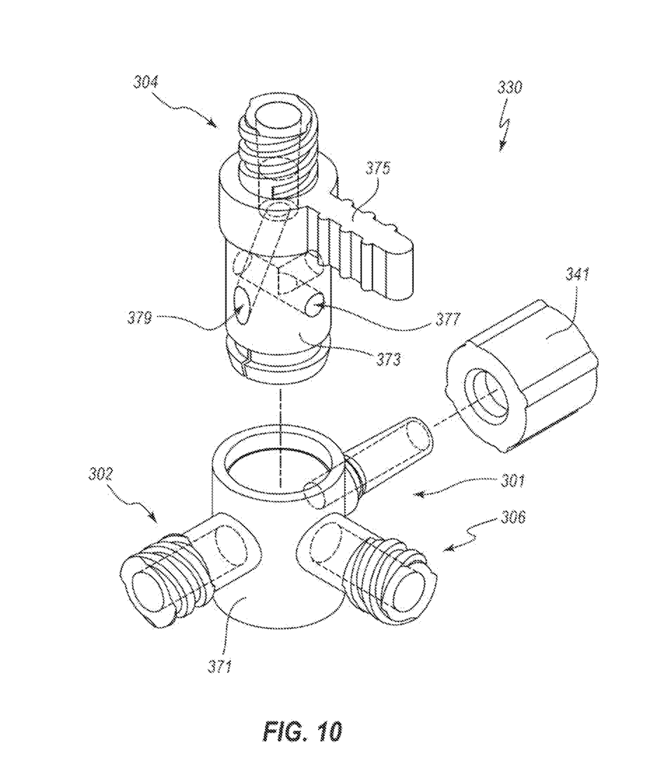

With reference to FIG. 10, the illustrated embodiment of the valve or stopcock 330 includes a housing member 371 and a rotatable insert member 373. The insert member 373 can include a handle 375, and can further define connecting channels 377, 379. The connecting channel 377 includes two branches that are perpendicular to each other and are in fluid communication with each other; one branch extends through an entirety of the insert 371, whereas the other extends through only a portion thereof.

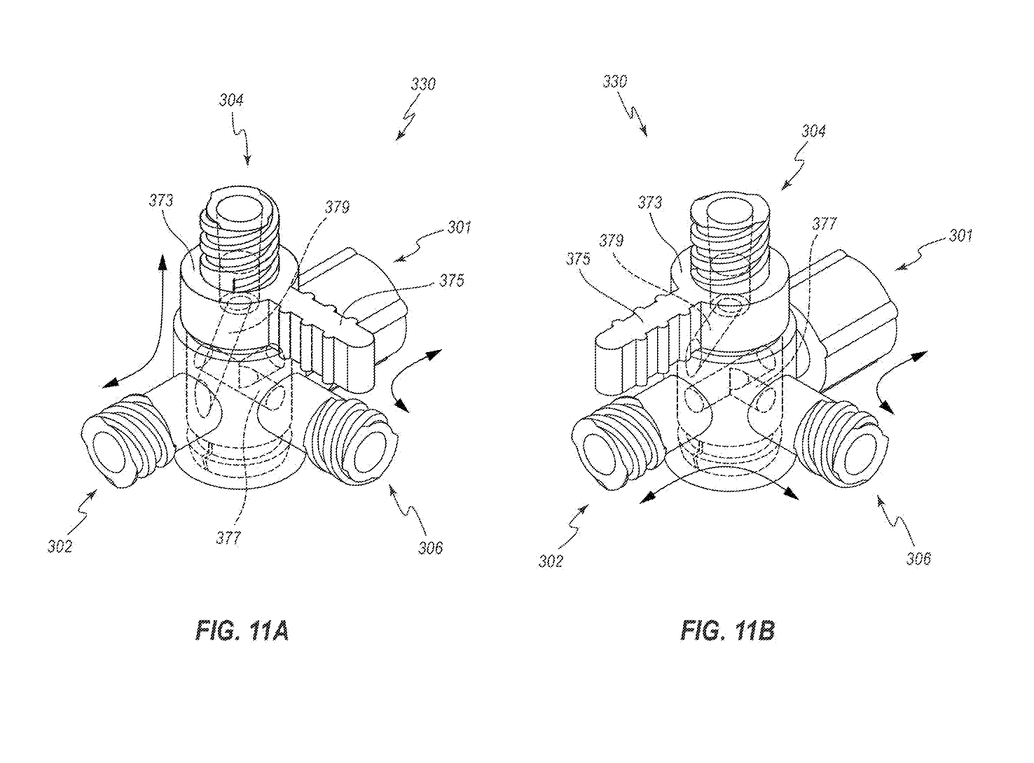

FIG. 11A illustrates a first operational state of the stopcock 330. In this operational state, the insert member 373 is oriented such that the connecting channel 377 provides fluid communication between the collection fluid path 301 and the sampling fluid path 306, and the connecting channel 379 provides fluid communication between the transfusion fluid path 302 and the delivery fluid path 304.

FIG. 11B illustrates a second operational state of the stopcock 330. In this operational state, the insert member 373 is oriented such that the connecting channel 377 provides fluid communication among the collection fluid path 301, the transfusion fluid path 304, and the sampling fluid path 306, whereas the connecting channel 379 isolates the delivery fluid path 304.

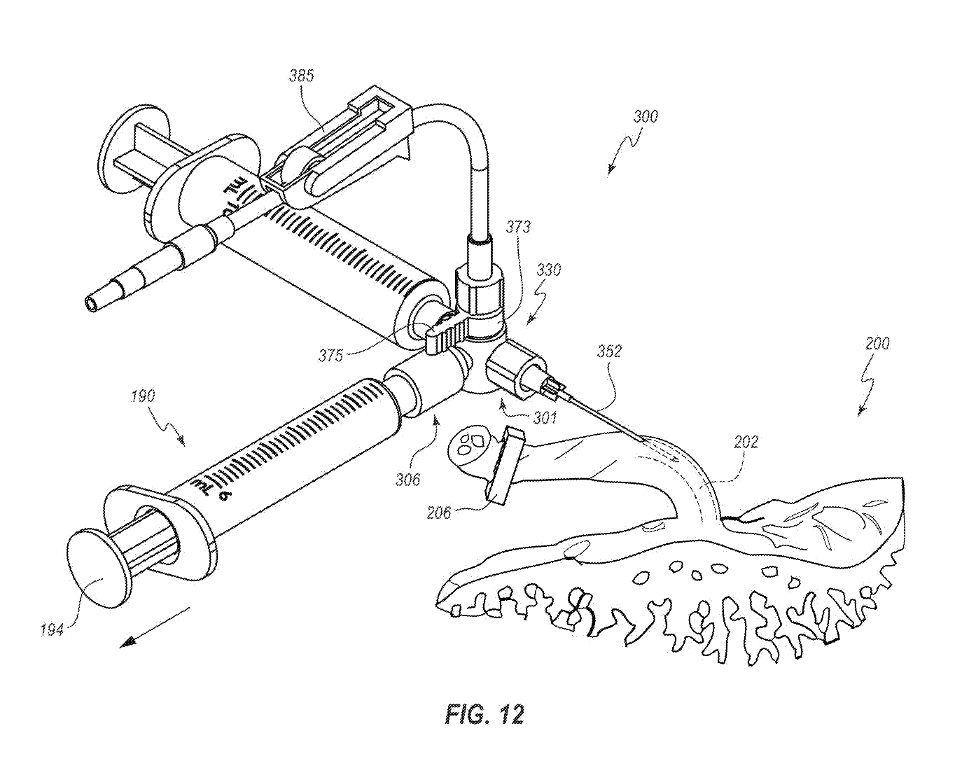

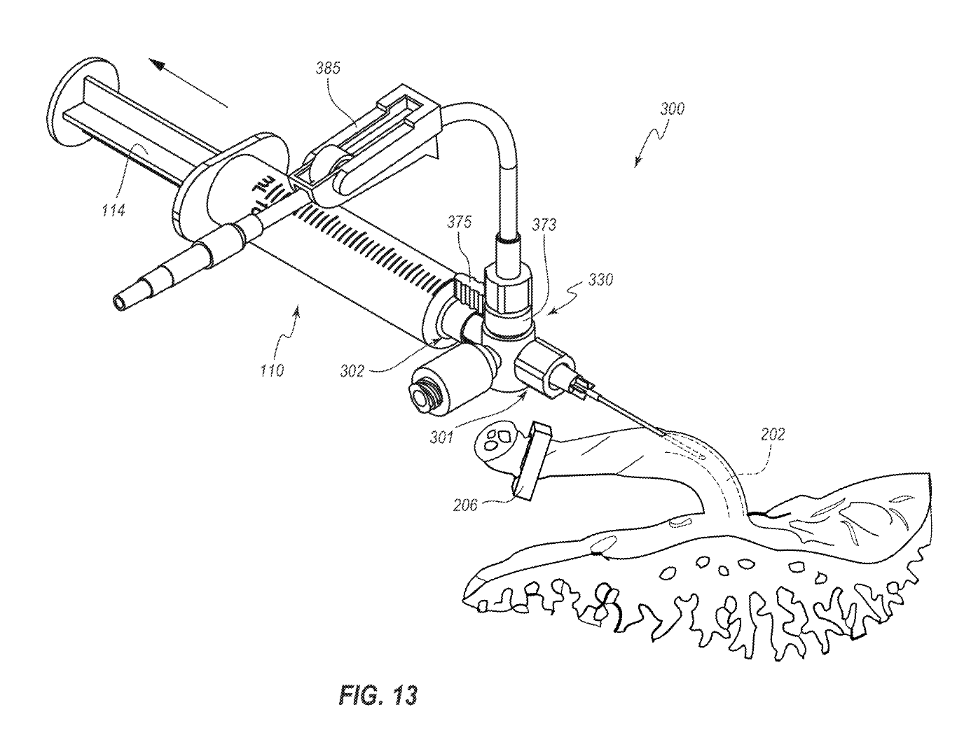

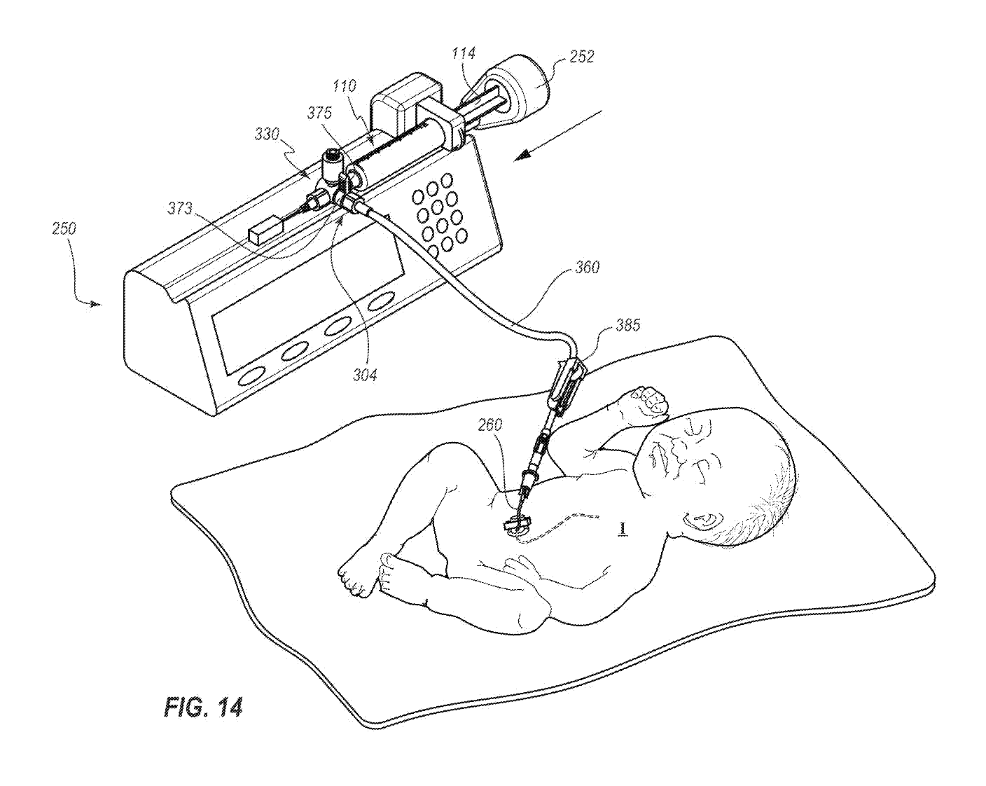

As illustrated in FIG. 12, when the assembly 300 is in a sample blood harvesting mode, the stopcock 330 can be in the first operational state (i.e., that of FIG. 11A). As illustrated in FIG. 13, when the assembly 300 is in a transfusion blood harvesting mode, the stopcock 330 can be in the second operational state (i.e., that of FIG. 11B). As illustrated in FIG. 14, when the assembly 300 is in a transfusion mode, the stopcock can again be in the first operational state (i.e., that of FIG. 11A). As can be appreciated, other arrangements of the stopcock 330 are possible.

In other embodiments, the assemblies 100, 300 are configured for operation in the transfusion blood harvesting mode and the transfusion mode. For example, the assemblies 100, 300 may not include the sample blood fluid paths 106, 306 (e.g., may not be configured for coupling with a sample syringe 190). Such embodiments may be configured primarily for transfusion applications.

In some embodiments the sample syringe 190 may be coupled with a first needle and the transfusion syringe 110 may be coupled with a second needle. Each syringe 110, 190 may be used to separately extract blood from the placenta 200. For example, the sample syringe 190 may be used to draw blood from the placenta 200 through a first access site in the placenta that is formed by the first needle, and the transfusion syringe 110 may be used to draw blood from the placenta 200 through a second access site in the placenta that is formed by the second needle. In various such embodiments, it may be desirable to form the access sites using the needles of the syringes 110, 190 at approximately the same time, or to ensure that both needles have been inserted into a blood vessel before any blood is drawn from the vessel, or to ensure that both needles have been inserted into a blood vessel before the needle of one of the syringes is removed from the vessel. This is because, in some instances, it may be difficult to withdraw blood from a blood vessel after a separate access site has been formed therein and is left open or unsealed, for example, due to bleeding and/or venting at the open or unsealed access site. Moreover, the clamped placental system may no longer be considered “closed” or sterile after a first needle has been used to access the system and is removed from the system. However, in other methods, one syringe 110, 190 may be used to collect blood and then removed from a vessel, and then another syringe 110, 190 may thereafter be inserted into the vessel and used to collect blood from the vessel.

In still further embodiments, a sample syringe 190 may be used on its own, without any transfusion procedures. For example, sample blood may be harvested using the sample syringe 190 or other blood collection device, and the blood can then be tested or otherwise processed. Such methods can prevent any sampling of blood directly from the infant, which may itself reduce or eliminate the need for a transfusion and/or save the infant from pain associated with a blood collection procedure.

As previously mentioned, while the drawings and written description have focused on illustrative devices, systems, and methods related to neonatal applications, it is to be understood that embodiments may be used in any other suitable context, such as contexts involving full-term infants. Moreover, it will be understood by those having skill in the art that changes may be made to the details of the above-described embodiments without departing from the underlying principles presented herein. For example, any suitable combination of various embodiments, or the features thereof, is contemplated. Additionally, blood receptacles other than those associated with syringes may be used in some embodiments.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

References to approximations are made throughout this specification, such as by use of the terms “about” or “approximately.” For each such reference, it is to be understood that, in some embodiments, the value, feature, or characteristic may be specified without approximation. For example, where qualifiers such as “about,” “substantially,” and “generally” are used, these terms include within their scope the qualified words in the absence of their qualifiers. For example, where the term “substantially planar” is recited with respect to a feature, it is understood that in further embodiments, the feature can have a precisely planar orientation.

Reference throughout this specification to “an embodiment” or “the embodiment” means that a particular feature, structure or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment.

The claims following this written disclosure are hereby expressly incorporated into the present written disclosure, with each claim standing on its own as a separate embodiment. This disclosure includes all permutations of the independent claims with their dependent claims.

Recitation in the claims of the term “first” with respect to a feature or element does not necessarily imply the existence of a second or additional such feature or element. Embodiments of the invention in which an exclusive property or privilege is claimed are defined as follows.

1. A method of providing autologous blood to an infant from the placenta of the infant, the method comprising:
providing an assembly that comprises a transfusion fluid path and a delivery fluid path, wherein the transfusion fluid path is connected to a first blood receptacle;
drawing blood from the placenta of an infant such that the blood passes through the transfusion fluid path and into the first blood receptacle;
moving blood from the first blood receptacle, through the delivery fluid path, and into the infant.

2. The method of claim 1, wherein the first blood receptacle comprises an anticoagulant therein prior to drawing blood into the first blood receptacle.

3. The method of claim 1, wherein the assembly further comprises a blood filter, the method further comprising passing blood through the blood filter.

4. The method of claim 3, wherein the blood filter is in the delivery fluid path.

5. The method of claim 1, wherein the delivery fluid path is connected to the transfusion fluid path, wherein said moving blood comprises moving blood from the first blood receptacle, through the transfusion fluid path, through the delivery fluid path, and into the infant.

6. The method of claim 1, wherein the assembly comprises a needle, the method further comprising inserting the needle into a blood vessel so as to draw blood therefrom, wherein at least a portion of the blood vessel extends along the placenta.

7. The method of claim 1, wherein the first blood receptacle is defined by a syringe that comprises a plunger, and wherein drawing blood from the placenta comprises pulling on the plunger.

8. The method of claim 7, wherein said moving blood from the first blood receptacle, through the delivery fluid path, and into the infant is performed by an automatic syringe pump.

9. The method of claim 1, wherein the assembly further comprises a second blood receptacle, the method further comprising drawing blood from the placenta into the second blood receptacle.

10. The method of claim 9, wherein drawing blood from the placenta into the second blood receptacle takes place before drawing blood from the placenta into the first blood receptacle.

11. The method of claim 9, further comprising removing the second blood receptacle from the assembly while leaving the first blood receptacle attached to the assembly.

12-33. (canceled)

34. A method of providing autologous blood to an infant from the infant's placenta, the method comprising:
providing an assembly that comprises a needle and a blood receptacle, wherein a plunger is coupled with the blood receptacle;
inserting the needle into a blood vessel so as to draw blood therefrom, wherein at least a portion of the blood vessel extends along the placenta;
drawing blood from a blood vessel into the blood receptacle by moving the plunger relative to the blood receptacle;
moving blood from the blood receptacle into the infant by moving the plunger relative to the blood receptacle.

35. The method of claim 34, wherein the blood receptacle comprises an anticoagulant.

36. The method of claim 34, wherein said moving blood from the blood receptacle into the infant is performed by an automatic pump.

37-42. (canceled)

43. A method of handling fetal blood, the method comprising:
providing a first blood receptacle that has a first plunger coupled thereto, wherein the first blood receptacle comprises an anticoagulant therein;
providing a second blood receptacle that has a second plunger coupled thereto;
drawing blood from a placenta into the first blood receptacle by moving the first plunger relative to the first blood receptacle; and
drawing blood from the placenta into the second blood receptacle by moving the second plunger relative to the second blood receptacle.

44. The method of claim 43, further comprising moving blood from the first blood receptacle into the infant by moving the first plunger relative to the first blood receptacle.

45. The method of claim 43, wherein a single assembly comprises the first blood receptacle and the second blood receptacle.

46. The method of claim 45, wherein the first blood receptacle is selectively isolated from the second blood receptacle.

47. The method of claim 45, wherein the assembly comprises a needle, the method further comprising inserting the needle into a blood vessel so as to draw blood therefrom, wherein at least a portion of the blood vessel extends along the placenta.

48. The method of claim 47, wherein blood is drawn from the blood vessel and into each of the first and second blood receptacles through the needle.

49-85. (canceled)
